SEQUENCE LISTING (1 sequence)

SEQ ID NO: 1 - DNA, 36 bp, synthetic construct
  source 1..36: mol_type = other DNA
  modified_base 1^2: mod_base = OTHER; note = phosphorothioate bond
  modified_base 2^3: mod_base = OTHER; note = phosphorothioate bond
  modified_base 34^35: mod_base = OTHER; note = phosphorothioate bond
  modified_base 35^36: mod_base = OTHER; note = phosphorothioate bond
  modified_base 1: mod_base = OTHER; note = phosphorylated
gtaagcgcttacgtaagcgcttacgtaagcgcttac

(19) United States
(12) Patent Application Publication
(10) Pub. No.: US 2025/0230455 A1
(43) Pub. Date: Jul. 17, 2025

(54) TARGETED DONOR DNA INSERTION AND INDEL EDITING OF PLANT GENES

(71) Applicant: INARI AGRICULTURE TECHNOLOGY, INC., Cambridge, MA (US)

(72) Inventors: Frédéric Van Ex, Kortrijk (BE); Hannes Bart Claeys, Ghent (BE)

(21) Appl. No.: 18/851,389

(22) PCT Filed: Mar. 27, 2023
(86) PCT No.: PCT/US2023/064994

§ 371 Date: Sep. 26, 2024

Related U.S. Application Data
(60) Provisional application No. 63/362,089, filed on Mar. 29, 2022.

(51) Int. Cl. C12N 15/82; C12N 9/22; C12N 15/11

(52) U.S. Cl. CPC C12N 15/8213; C12N 9/22; C12N 15/11; C12N 2310/20

(57) ABSTRACT

The disclosure relates to methods of producing genome edited plant cells, genome edited plant calli, and genome edited plants with genome edits at multiple genomic sites. The disclosure also relates to systems and kits for producing genome edited plant cells with genome edits at multiple genomic sites. Disclosed methods include those where a guide RNA (gRNA) directed to a first genomic DNA site, a donor DNA template polynucleotide, and a polynucleotide comprising a DNA molecule encoding a second gRNA directed to a second genomic DNA site are introduced into a plant cell and genome edited cell having an insertion of the donor DNA template polynucleotide or fragment thereof in the first genomic DNA site and a DNA modification at the second genomic DNA site is selected.

### CROSS-REFERENCE TO RELATED APPLICATIONS

This international application claims the benefit of U.S. Provisional Patent Application Ser. No. 63/362,089, filed Mar. 29, 2022, which is incorporated herein by reference in its entirety.

### REFERENCE TO SEQUENCE LISTING SUBMITTED ELECTRONICALLY

The instant application contains a Sequence Listing which has been submitted electronically in XML format and is herein incorporated by reference in its entirety. Said XML copy, created on Mar. 27, 2023, is named “P13840WO00_SequenceListing.xml” and is 4,775 bytes in size.

### BACKGROUND

Methods of using CRISPR, Zinc Finger Nuclease, and Transcription activator like effector Nuclease (TALEN) technology for genome editing in plants are disclosed in US 20150082478, US 2015/0059010A1, and Bortesi et al., 2015, Biotechnology Advances, pp. 41-52, Vol. 33, No. 1. Although genome editing techniques are available for producing targeted insertions, substitutions, and deletions of sequences into plants, there remains a need for improved methods to efficiently edit multiple genomic sites of a plant that minimize the number of editing events which need to be screened for the presence of the desired gene edits. In particular, methods for efficient and one step introduction for one type of genome edit into one site in the genome and another type of genome edit into another site in the genome are needed.

### SUMMARY

Methods of producing a genome edited plant cell comprising: (a) introducing into a plant cell a first guide RNA (gRNA) directed to a first genomic DNA site, a donor DNA template polynucleotide, and a polynucleotide comprising a DNA molecule encoding a second gRNA directed to a second genomic DNA site; wherein the plant cell comprises at least one Cas nuclease and/or one or more polynucleotides encoding the Cas nuclease(s); and (b) selecting for the genome edited plant cell, wherein the genome edited plant cell comprises a first insertion of the donor DNA template polynucleotide or fragment thereof in the first genomic DNA site and a DNA modification at the second genomic DNA site comprising a second insertion, a deletion, and/or a substitution of one or more nucleotides, and wherein the second insertion does not comprise the donor DNA template polynucleotide or fragment thereof are provided.

Plant cell genome editing systems comprising a target plant cell containing a first guide RNA (gRNA) directed to a first genomic DNA site, a donor DNA template polynucleotide, a polynucleotide comprising a DNA molecule encoding a second gRNA directed to a second genomic DNA site, and at least one Cas nuclease and/or one or more polynucleotides encoding the Cas nuclease(s), wherein the system provides a genome edited plant cell comprising a first insertion of the donor DNA template polynucleotide or fragment thereof in the first genomic DNA site and a DNA modification at the second genomic DNA site comprising a second insertion, a deletion, and/or a substitution of one or more nucleotides, wherein the second insertion does not comprise the donor DNA template polynucleotide or fragment thereof, are provided.

Kits comprising: (a) a first guide RNA (gRNA) directed to a first genomic DNA site in a plant cell and a donor DNA template polynucleotide; and (b) a polynucleotide comprising a DNA molecule encoding a second gRNA directed to a second genomic DNA site in a plant cell are provided.

### DETAILED DESCRIPTION

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

As used herein, the term “expression” refers to the production of a functional end-product (e.g., an mRNA, guide RNA, or a protein) in either precursor or mature form.

As used herein, the terms “include,” “includes,” and “including” are to be construed as at least having the features to which they refer while not excluding any additional unspecified features.

As used herein, the term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell and includes reference to the transient provision of a nucleic acid or protein to the cell. Introduced includes reference to stable or transient transformation methods. Thus, “introduced” in the context of inserting a nucleic acid fragment (e.g., a recombinant DNA construct/expression construct) into a cell, means “transfection” or “electroporation” or “transformation” or “transduction” and includes reference to the incorporation of a nucleic acid fragment into a eukaryotic or prokaryotic cell where the nucleic acid fragment may be incorporated into the genome of the cell (e.g., nuclear chromosome, plasmid, plastid, chloroplast, or mitochondrial DNA), converted into an autonomous replicon, or transiently expressed (e.g., transfected mRNA).

The term “isolated” as used herein means having been removed from its natural environment.

As used herein, the phrase “operably linked” refers to a juxtaposition wherein the components so described are in a relationship permitting them to function in their intended manner. For instance, a promoter is operably linked to a coding sequence if the promoter affects its transcription or expression.

As used herein, the term “plant” includes a whole plant and any descendant, cell, tissue, or part of a plant. The term “plant parts” include any part(s) of a plant, including, for example and without limitation: seed (including mature seed and immature seed); a plant cutting; a plant cell; a plant cell culture; or a plant organ (e.g., pollen, embryos, flowers, fruits, shoots, leaves, roots, stems, and explants). A plant tissue or plant organ may be a seed, protoplast, callus, or any other group of plant cells that is organized into a structural or functional unit. A plant cell or tissue culture may be capable of regenerating a plant having the physiological and morphological characteristics of the plant from which the cell or tissue was obtained, and of regenerating a plant having substantially the same genotype as the plant. Regenerable cells in a plant cell or tissue culture may be embryos, protoplasts, meristematic cells, callus, pollen, leaves, anthers, roots, root tips, silk, flowers, kernels, ears, cobs, husks, or stalks. In contrast, some plant cells are not capable of being regenerated to produce plants and are referred to herein as “non-regenerable” plant cells.

As used herein, “selecting” is understood as identifying and isolating or enriching for one or more plant cells, plant callus, or plants having a desired characteristic or property of interest. Desired characteristics or properties of interest include an insertion of a donor DNA template polynucleotide or fragment thereof at one or more genomic sites, an INDELS at one or more genomic sites, a selectable marker, a phenotype conferred by the insertion(s) or INDELS, and/or a change in expression of one or more plant genes resulting from the insertion(s) or INDELS.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

Methods, systems, and kits which allow efficient genome editing of a multiple genomic DNA sites in plant cells are provided herein. In certain embodiments, the methods, systems, and kits provide for introduction of distinct types of genomic edits at two or more distinct genomic loci in the genome of a target plant cell. In certain embodiments, the methods and systems can comprise a step for introduction of gene-editing molecules to provide the distinct genomic edits at the distinct genomic sites followed by a step for selection of a plant cell having the desired genomic edits at the distinct genomic sites. Examples of distinct genome edits provided by the methods, systems, and kits provide herein can include a first genome edit comprising a first insertion of a donor DNA template polynucleotide or fragment thereof in a first genomic site and a second distinct genomic edit comprising a second insertion, a deletion, and/or a substitution (INDELS) of one or more nucleotides in a second genomic site. In such methods, systems, and kits, the distinct types of genomic edits which are introduced allow and/or expedite recovery of plant cells having the desired distinct genomic edits (e.g., DNA template polynucleotide or fragment thereof in a first genomic site and an INDELS in the second genomic site) without having to first introduce and select for a plant cell or plant having the first desired genomic edit(s) and then subsequently introduce the second desired genomic edit(s) into the selected a plant cell or plant having the first desired genomic edit(s).

Methods, systems, and kits provided herein can provide for distinct types of genomic edits at two or more genomic locations by simultaneous or staggered introduction of gene editing molecules into a target plant cell lacking the desired genomic edits. In certain embodiments, the distinct types of genomic edits at two or more genomic locations are attained by introducing the gene editing regents in a single step by way of a single type of introduction method (e.g., biolistic-, transfection-, or electroporation-mediated delivery of gene editing regents comprising proteins, ribonucleoproteins, and/or polynucleotides, or bacterially-mediated transformation of polynucleotides encoding proteins and/or polynucleotides). An example of a single step introduction includes biolistic-, transfection-, or electroporation-mediated delivery of gene-editing molecules comprising a gRNA directed to a first distinct genomic site, a donor DNA template polynucleotide, and a DNA molecule encoding at least a gRNA directed to a second genomic site. An example of a single step introduction also includes biolistic-, transfection-, or electroporation-mediated delivery of gene-editing molecules comprising a gRNA directed to a first distinct genomic site and a donor DNA template polynucleotide followed by a second biolistic-, transfection-, or electroporation-mediated delivery of a gene-editing molecule(s) comprising a DNA molecule encoding at least a gRNA directed to a second genomic site, wherein the DNA molecule can optionally further comprise sequences encoding one or more additional gene-editing molecules (e.g., a Cas nuclease). In certain embodiments, the distinct types of genomic edits at two or more genomic locations are obtained by introducing the gene editing regents by way of two types of introduction methods (e.g., biolistic-, electroporation-, or transfection-mediated delivery of gene editing molecules comprising proteins, ribonucleoproteins, and/or polynucleotides, followed by bacterially-mediated transformation of polynucleotides encoding proteins and/or polynucleotides). In certain embodiments, the distinct types of genomic edits at two or more genomic locations are obtained by introducing the gene editing regents by way of staggered introduction of gene editing molecules comprising initial introduction of a first set of gene editing molecules directed to a first genomic site (gene-editing molecules comprising a gRNA directed to a first distinct genomic site and a donor DNA template polynucleotide) followed by introduction of a second set of gene editing molecules (e.g., a polynucleotide encoding a second gRNA directed to a second genomic location). In certain embodiments, the staggered introduction of the gene editing molecules can be either be by way of the same introduction method (e.g., staggered biolistic-, electroporation-, or transfection-mediated delivery of gene editing molecules) or by way of different introduction methods (e.g., biolistic-mediated delivery of gene editing molecules followed by bacterially-mediated delivery of gene editing molecules). In certain embodiments, such gene editing molecules can comprise a gRNA directed to a first distinct genomic site either alone or complexed with a Cas nuclease in a ribonuclear protein (e.g., an RNP), a donor DNA template polynucleotide, and a DNA molecule encoding a gRNA directed to a second genomic site, as well as Cas nuclease(s) and/or a polynucleotide encoding same if the plant cell lacks a Cas nuclease. In certain embodiments, a first distinct genomic DNA edit comprising an insertion of a donor DNA template polynucleotide or fragment thereof at a first genomic location (e.g., non-homologous end joining mediated (NHEJ)-or homology directed repair (HDR)-mediated insertion of a DNA molecule comprising an enhancer element to upregulate the transcription of a target gene) and a second distinct genomic DNA edit comprising an INDELS (insert, deletion, and/or substitution) at a second genomic location (e.g., altering a coding or non-coding region of a target gene to generate a loss-of-function allele; altering a negative-regulatory element to generate a gain of function allele) are generated. In certain aforementioned embodiments, the target plant cell can comprise a Cas nuclease or a polynucleotide encoding a Cas nuclease prior to introduction of the gene editing molecules. In certain aforementioned embodiments that include those where the target plant comprises a Cas nuclease or a polynucleotide encoding a Cas nuclease, gene editing molecules comprising a polynucleotide encoding a Cas nuclease can also be introduced into the plant cell. In certain embodiments, a first Cas nuclease which recognizes the first gRNA directed to the first genomic site is present in the target plant cell or is provided (e.g., as an RNP complexed with the first gRNA) and a second Cas nuclease which recognizes the second gRNA directed to the second genomic site is provided (e.g., as a polynucleotide which encodes the second Cas nuclease). In certain embodiments, the first Cas nuclease does not recognize the second gRNA and the second Cas nuclease does not recognize the first gRNA (e.g., the first Cas nuclease(s) comprises a Type II Cas nuclease and the second Cas nuclease is a Type V Cas nuclease.)

Methods, systems, and kits provided herein can direct or bias the formation of intended distinct genomic edits (e.g., a donor DNA template polynucleotide or fragment thereof insertion genomic edit or an INDELS genomic edit) to their intended distinct genomic sites. In certain embodiments, the methods, systems, and kits provided herein can provide significant decreases in the number of candidate plant cells subjected to genome editing that need to be screened to obtain genome edited plant cells with the two or more intended genomic edits (e.g., a insertion of a donor DNA template polynucleotide or fragment thereof at a first genomic location and an INDELS at a second genomic location) in comparison to standard methods, systems, and kits (e.g., where both a first and a second distinct gRNA are provided simultaneously as RNA molecules and/or by a DNA molecule encoding the gRNAs). In certain embodiments, the methods, systems, and kits provided herein can provide significant decreases in the time required to obtain the intended genomic edits (e.g., a insertion of a donor DNA template polynucleotide or fragment thereof at a first genomic location and an INDELS at a second genomic location) in comparison to standard methods, systems, and kits (e.g., where gene edits are introduced independently in plant cells which are subsequently regenerated into plants and crossed or where a first edit is introduced and recovered in a plant cell with a first genomic edit and a second edit is subsequently introduced into the plant cell with a first genomic edit).

The methods, systems, and kits provided herein can provide for selection of plant cells, plant callus, and/or plants comprising the desired distinct genomic edits to the distinct genomic sites. In certain embodiments, the selections are performed on populations of plant cells, plant calli, or plants comprising: (i) plant cells, plant calli, or plants comprising all of the desired genomic edits; (ii) plant cells, plant calli, or plants comprising a subset of the desired genomic edits; and/or (iii) plant cells, plant calli, or plants lacking the desired genomic edits. Plant cells, plant calli, or plants comprising the desired genomic edits can be selected from populations of plant cells, plant calli, or plants by assaying plant cells, plant calli, or plants where the gene editing molecules directed to the first and second target genomic loci had been introduced. Such assaying can comprise a phenotypic assay, a nucleic acid amplification-based assay, a nucleic acid hybridization-based assay, and/or a sequencing-based assay for the desired distinct genomic edits to the distinct genomic sites, wherein the assaying comprises a phenotypic assay, a nucleic acid amplification-based assay, a nucleic acid hybridization-based assay, and/or a sequencing-based assay for the insertion and/or the DNA modification. In certain embodiments, the phenotypic assay can assay for one or more traits conferred by the genome edits. Phenotypic traits conferred by desired genome edits which can be assayed include improved yield, improved food and/or feed characteristics (e.g., improved oil, starch, protein, or amino acid quality or quantity), improved nitrogen use efficiency, improved biofuel use characteristics (e.g., improved ethanol production), male sterility/conditional male sterility systems (e.g., by targeting endogenous MS26, MS45 and MSCA1 genes), herbicide tolerance (e.g., by targeting endogenous ALS, EPSPS, HPPD, or other herbicide target genes), delayed flowering, non-flowering, increased biotic stress resistance (e.g., resistance to insect, nematode, bacterial, or fungal damage), increased abiotic stress resistance (e.g., resistance to drought, cold, heat, metal, or salt), enhanced lodging resistance, enhanced growth rate, enhanced biomass, enhanced tillering, enhanced branching, delayed flowering time, delayed senescence, increased flower number, improved architecture for high density planting, improved photosynthesis, increased root mass, increased cell number, improved seedling vigor, improved seedling size, increased rate of cell division, improved metabolic efficiency, and increased meristem size in comparison to a control plant lacking the targeted genetic change. Non-limiting examples of regulatory sequences which can be inserted into endogenous plant genes with gene editing molecules to effect targeted genetic changes which confer useful phenotypes include those set forth in U.S. Pat. No. 11,198,885 and US Patent Application Publication 20190352655, which are incorporated herein by reference in their entireties. Non-limiting examples of target genes in crop plants including corn and soybean which can be subjected to targeted genetic changes which confer useful phenotypes include those set forth in U.S. patents application Nos. 20190352655, 20200199609, 20200157554, and 20200231982, which are each incorporated herein in their entireties; and Zhang et al. (Genome Biol. 2018; 19:210). Phenotypic assays for genomic edits can also include assays for changes in the expression of target genes (e.g., by hybridization and/or amplification-based gene expression assays including Reverse Transcriptase-PCR analyses). Presence of edits of the target gene by any of various molecular assays, including, e. g., T7E1 assay, fragment analyzer assay, Sanger sequencing, enrichment of edited amplicons by restriction digest, quantitative real time PCR (e.g., Li, R. et al. Food Control 112:107088; doi: 10.1016/j.foodcont.2020.107088) and NGS amplicon sequencing (e.g., Glenn, T C et al. PeerJ 7:e7786. doi: 10.7717/peerj.7786). Various methods for detecting genome edits in plants comprising PCR, digital PCR, sequencing, isothermal DNA detection, and CRISPR-Cas-mediated edit detection have been disclosed (Shillito, R. D., et al. In Vitro Cell.Dev.Biol.-Plant 57, 595-608 (2021). doi: 10.1007/s11627-021-10214-z) and can be adapted for detection of desired genomic edits.

In certain embodiments, methods provided herein can include the additional step of growing or regenerating a genome edited plant from a genome edited plant cell or a genome edited plant callus. In certain embodiments, callus is produced from the plant cell, and plantlets and plants produced from such callus. In other embodiments, whole seedlings or plants are grown directly from the plant cell without a callus stage. Methods for obtaining regenerable plant structures and regenerating plants from plant cells or regenerable plant structures can be adapted from published procedures (Roest and Gilissen, Acta Bot. Neerl., 1989, 38(1), 1-23; Bhaskaran and Smith, Crop Sci. 30(6): 1328-1337; Ikeuchi et al., Development, 2016, 143:1442-1451). Methods for obtaining regenerable plant structures and regenerating plants from plant cells or regenerable plant structures can also be adapted from US Patent Application Publication No. 20170121722, which is incorporated herein by reference in its entirety and specifically with respect to such disclosure.

Gene editing molecules of use in methods provided herein include molecules capable of introducing a double-strand break (“DSB”) or single-strand break (“SSB”) in double-stranded DNA, such as in genomic DNA or in a target gene located within the genomic DNA as well as accompanying guide RNA or donor DNA template polynucleotides. Examples of such gene editing molecules include: (a) a nuclease comprising an RNA-guided nuclease, an RNA-guided DNA endonuclease or RNA directed DNA endonuclease (RdDe), a class 1 CRISPR type nuclease system, a Type II Cas nuclease, a Cas9, a nCas9 nickase, a Type V Cas nuclease, a Cas12a nuclease, a nCas12a nickase, a Cas12d (CasY), a Cas12e (CasX), a Cas12b (C2c1), a Cas12c (C2c3), a Cas12i, a Cas12j, a Cas14, an engineered nuclease, a codon-optimized nuclease, a zinc-finger nuclease (ZFN) or nickase, a transcription activator-like effector nuclease (TAL-effector nuclease or TALEN) or nickase (TALE-nickase), an Argonaute, and a meganuclease or engineered meganuclease; (b) a polynucleotide encoding one or more nucleases capable of effectuating site-specific alteration (including introduction of a DSB or SSB) of a target nucleotide sequence; (c) a guide RNA (gRNA) for an RNA-guided nuclease, or a DNA encoding a gRNA for an RNA-guided nuclease; (d) donor DNA template polynucleotides; and (e) other DNA templates (dsDNA, ssDNA, or combinations thereof) suitable for insertion at a break in genomic DNA (e.g., by non-homologous end joining (NHEJ) or microhomology-mediated end joining (MMEJ).

CRISPR-type genome editing can be adapted for use in the plant cells and methods provided herein in several ways. CRISPR elements, e.g., gene editing molecules comprising CRISPR endonucleases and CRISPR guide RNAs including single guide RNAs or guide RNAs in combination with tracrRNAs or scoutRNA, or polynucleotides encoding the same, are useful in effectuating genome editing without remnants of the CRISPR elements or selective genetic markers occurring in progeny. In certain embodiments, the CRISPR elements are provided directly to the eukaryotic cell (e.g., plant cells), systems, methods, and compositions as isolated molecules, as isolated or semi-purified products of a cell free synthetic process (e.g., in vitro translation), or as isolated or semi-purified products of in a cell-based synthetic process (e.g., such as in a bacterial or other cell lysate). In certain embodiments, one or more CRISPR endonucleases with unique PAM recognition sites can be used. Guide RNAs (sgRNAs or crRNAs and a tracrRNA) to form an RNA-guided endonuclease/guide RNA complex which can specifically bind sequences in the gDNA target site that are adjacent to a protospacer adjacent motif (PAM) sequence. The type of RNA-guided endonuclease typically informs the location of suitable PAM sites and design of crRNAs or sgRNAs. G-rich PAM sites, e.g., 5′-NGG are typically targeted for design of crRNAs or sgRNAs used with Cas9 proteins. Examples of PAM sequences include 5′-NGG (Streptococcus pyogenes), 5′-NNAGAA (Streptococcus thermophilus CRISPR1), 5′-NGGNG (Streptococcus thermophilus CRISPR3), 5′-NNGRRT or 5′-NNGRR (Staphylococcus aureus Cas9, SaCas9), and 5′-NNNGATT (Neisseria meningitidis). T-rich PAM sites (e.g., 5′-TTN or 5′-TTTV, where “V” is A, C, or G) are typically targeted for design of crRNAs or sgRNAs used with Cas12a proteins. In some instances, Cas12a can also recognize a 5′-CTA PAM motif. Other examples of potential Cas12a PAM sequences include TTN, CTN, TCN, CCN, TTTN, TCTN, TTCN, CTTN, ATTN, TCCN, TTGN, GTTN, CCCN, CCTN, TTAN, TCGN, CTCN, ACTN, GCTN, TCAN, GCCN, and CCGN (wherein N is defined as any nucleotide). Cpf1 endonuclease and corresponding guide RNAs and PAM sites are disclosed in US Patent Application Publication 2016/0208243 A1, which is incorporated herein by reference for its disclosure of DNA encoding Cpf1 endonucleases and guide RNAs and PAM sites. Introduction of one or more of a wide variety of CRISPR guide RNAs that interact with CRISPR endonucleases integrated into a plant genome or otherwise provided to a plant is useful for genetic editing for providing desired phenotypes or traits, for trait screening, or for gene editing mediated trait introgression (e.g., for introducing a trait into a new genotype without backcrossing to a recurrent parent or with limited backcrossing to a recurrent parent). Multiple endonucleases can be provided in expression cassettes with the appropriate promoters to allow multiple genome site editing.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1. Other CRISPR nucleases useful for editing genomes include Cas12b and Cas12c (see Shmakov et al. (2015) Mol. Cell, 60:385-397; Harrington et al. (2020) Molecular Cell doi: 10.1016/j.molcel.2020.06.022) and CasX and CasY (see Burstein et al. (2016) Nature, doi: 10.1038/nature21059; Harrington et al. (2020) Molecular Cell doi: 10.1016/j.molcel.2020.06.022), or Cas12j (Pausch et al, (2020) Science 10.1126/science.abb1400). Plant RNA promoters for expressing CRISPR guide RNA and plant codon-optimized CRISPR Cas9 endonuclease are disclosed in International Patent Application PCT/US2015/018104 (published as WO 2015/131101 and claiming priority to U.S. Provisional Patent Application 61/945,700). Methods of using CRISPR technology for genome editing in plants are disclosed in US Patent Application Publications US 2015/0082478A1 and US 2015/0059010A1 and in International Patent Application PCT/US2015/038767 A1 (published as WO 2016/007347 and claiming priority to U.S. Provisional Patent Application 62/023,246). All of the patent publications referenced in this paragraph are incorporated herein by reference in their entireties.

In certain embodiments, an RNA-guided endonuclease that leaves a blunt end following cleavage of the target site is used. Blunt-end cutting RNA-guided endonucleases include Cas9, Cas12c, and Cas 12h (Yan et al., 2019). In certain embodiments, an RNA-guided endonuclease that leaves a staggered single stranded DNA overhanging end following cleavage of the target site following cleavage of the target site is used. Staggered-end cutting RNA-guided endonucleases include Cas12a, Cas12b, and Cas12e.

The methods can also use sequence-specific endonucleases or sequence-specific endonucleases and guide RNAs that cleave a single DNA strand in a dsDNA target site. Such cleavage of a single DNA strand in a dsDNA target site is also referred to herein and elsewhere as “nicking” and can be affected by various “nickases” or systems that provide for nicking. Nickases that can be used include nCas9 (Cas9 comprising a D10A amino acid substitution), nCas12a (e.g., Cas12a comprising an R1226A amino acid substitution; Yamano et al., 2016), Cas12i (Yan et al. 2019), a zinc finger nickase e.g., as disclosed in Kim et al., 2012), a TALE nickase (e.g., as disclosed in Wu et al., 2014), or a combination thereof. In certain embodiments, systems that provide for nicking can comprise a Cas nuclease (e.g., Cas9 and/or Cas12a) and guide RNA molecules that have at least one base mismatch to DNA sequences in the target editing site (Fu et al., 2019). In certain embodiments, genome modifications can be introduced into the target editing site by creating single stranded breaks (i.e., “nicks”) in genomic locations separated by no more than about 10, 20, 30, 40, 50, 60, 80, 100, 150, or 200 base pairs of DNA. In certain illustrative and non-limiting embodiments, two nickases (i.e., a CAS nuclease which introduces a single stranded DNA break including nCas9, nCas12a, Cas12i, Cas 12j, zinc finger nickases, TALE nickases, combinations thereof, and the like) or nickase systems can directed to make cuts to nearby sites separated by no more than about 10, 20, 30, 40, 50, 60, 80 or 100 base pairs of DNA. In instances where an RNA guided nickase and an RNA guide are used, the RNA guides are adjacent to PAM sequences that are sufficiently close (i.e., separated by no more than about 10, 20, 30, 40, 50, 60, 80, 100, 150, or 200 base pairs of DNA). For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least 16 or 17 nucleotides of gRNA sequence are required by Cas9 for DNA cleavage to occur; for Cpf1 at least 16 nucleotides of gRNA sequence are needed to achieve detectable DNA cleavage and at least 18 nucleotides of gRNA sequence were reported necessary for efficient DNA cleavage in vitro; see Zetsche et al. (2015) Cell, 163:759-771. In practice, guide RNA sequences are generally designed to have a length of 17-24 nucleotides (frequently 19, 20, or 21 nucleotides) and exact complementarity (i.e., perfect base-pairing) to the targeted gene or nucleic acid sequence; guide RNAs having less than 100% complementarity to the target sequence can be used (e.g., a gRNA with a length of 20 nucleotides and 1-4 mismatches to the target sequence) but can increase the potential for off-target effects. The design of effective guide RNAs for use in plant genome editing is disclosed in US Patent Application Publication 2015/0082478 A1, the entire specification of which is incorporated herein by reference. More recently, efficient gene editing has been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing); see, for example, Cong et al. (2013) Science, 339:819-823; Xing et al. (2014) BMC Plant Biol., 14:327-340. Chemically modified sgRNAs have been demonstrated to be effective in genome editing; see, for example, Hendel et al. (2015) Nature Biotechnol., 985-991. The design of effective gRNAs for use in plant genome editing is disclosed in US Patent Application Publication 2015/0082478 A1, the entire specification of which is incorporated herein by reference.

The genome-editing methods, systems, and kits provided herein can include a donor DNA template polynucleotide. In certain embodiments, at least one double-stranded break (DSB) is effected at a precisely determined site in the plant genome, for example by means of an RNA-guided nuclease and guide RNAs, and a nucleotide sequence encoded by a donor polynucleotide is heterologously integrated at the site of the DSB (or between two DSBs). In embodiments, the donor polynucleotide includes single-stranded DNA, optionally including chemical modifications. In other embodiments, the donor polynucleotide includes double-stranded DNA, optionally including chemical modifications. In some embodiment the donor polynucleotide includes both DNA and RNA, for example as a duplex formed by a DNA strand and an RNA strand.

In embodiments, the donor DNA template polynucleotide includes chemically modified nucleotides (see, e.g., the various modifications of internucleotide linkages, bases, and sugars described in Verma and Eckstein (1998) Annu. Rev. Biochem., 67:99-134); in embodiments, the naturally occurring phosphodiester backbone of the donor DNA template polynucleotide is partially or completely modified with phosphorothioate, phosphorodithioate, or methylphosphonate internucleotide linkage modifications, or the donor DNA template polynucleotide includes modified nucleoside bases or modified sugars, or the donor DNA template polynucleotide is labelled with a fluorescent moiety (e.g., fluorescein or rhodamine or a fluorescent nucleoside analogue) or other detectable label (e.g., biotin or an isotope). In another embodiment, the donor DNA template polynucleotide contains secondary structure that provides stability or acts as an aptamer.

Donor DNA template polynucleotides used in the methods, systems, and kits provided herein include DNA molecules comprising, from 5′ to 3′, a first homology arm, a replacement DNA, and a second homology arm, wherein the homology arms containing sequences that are partially or completely homologous to genomic DNA (gDNA) sequences flanking a target site-specific endonuclease cleavage site in the gDNA. In certain embodiments, the replacement DNA can comprise an insertion, deletion, or substitution of 1 or more DNA base pairs relative to the target gDNA. In one embodiment, the donor DNA template polynucleotide is double-stranded and perfectly base-paired through all or most of its length, with the possible exception of any unpaired nucleotides at either terminus or both termini. In another embodiment, the donor DNA template polynucleotide is double-stranded and includes one or more non-terminal mismatches or non-terminal unpaired nucleotides within the otherwise double-stranded duplex. In an embodiment, the donor DNA template polynucleotide that is integrated at the site of at least one double-strand break (DSB) includes between 2-20 nucleotides in one (if single-stranded) or in both strands (if double-stranded), e. g., 1, 2, 3, 4, 5, 6, 7, 8, 9, 10, 11, 12, 13, 14, 15, 16, 17, 18, 19, or 20 nucleotides on one or on both strands, each of which can be base-paired to a nucleotide on the opposite strand of the targeted integration site (in the case of a perfectly base-paired double-stranded polynucleotide molecule). Such donor DNA templates can be integrated in genomic DNA containing blunt and/or staggered double stranded DNA breaks by homology-directed repair (HDR) or microhomology-mediated end joining (MMEJ). In certain embodiments, a donor DNA template homology arm can be about 20, 50, 100, 200, 400, or 600 to about 800, or 1000 base pairs in length. In certain embodiments, a donor DNA template polynucleotide can be delivered to a plant cell in a circular (e.g., a plasmid or a viral vector including a geminivirus vector) or a linear DNA molecule. In certain embodiments, a circular or linear DNA molecule that is used can comprise a modified donor DNA template polynucleotide comprising, from 5′ to 3′, a first copy of the target sequence-specific endonuclease cleavage site sequence, the first homology arm, the replacement DNA, the second homology arm, and a second copy of the target sequence-specific endonuclease cleavage site sequence. In other embodiments, donor DNA template polynucleotides suitable for NHEJ insertion will lack homology arms that are partially or completely homologous to genomic DNA (gDNA) sequences flanking a target site-specific endonuclease cleavage site in the gDNA. Donor DNA template polynucleotides can be synthesized either chemically or enzymatically (e.g., in a polymerase chain reaction (PCR)).

Substitution is optionally by way of homology directed repair (HDR) wherein a donor DNA template polynucleotide comprising replacement DNA used to substitute genomic DNA targeted by a double-stranded break inducing agent. In certain embodiments, HDR-mediated substitution is facilitated by expressing an exonuclease (e.g., a bacteriophage lambda exonuclease), a single-stranded DNA annealing protein (SSAP; e.g., bacteriophage lambda beta SSAP protein), and a single stranded DNA binding protein (SSB; e.g., E. coli SSB) essentially as set forth in US Patent Application Publication 20200407754, which is incorporated herein by reference in its entirety. A DNA sequence encoding a localization signal (NLS; e.g., tobacco c2 NLS) is fused in-frame to the DNA sequences encoding the exonuclease, the SSAP protein, and the SSB. In certain embodiments, a DNA sequence encoding the c2 NLS-Exo, c2 NLS lambda beta SSAP, and c2 NLS-SSB fusion proteins that are set forth in SEQ ID NO: 135, SEQ ID NO: 134, and SEQ ID NO: 133 of US Patent Application Publication 20200407754, respectively, and incorporated herein by reference in its entirety is used. In certain embodiments, DNA sequences encoding the NLS-Exo, NLS-SSAP, and NLS-SSB fusion proteins are operably linked to a promoter (e.g., OsUBI1, ZmUBI1, OsACT promoter) and a polyadenylation site (e.g., OsUbi1, ZmUBI1, OsACT polyadenylation site), to provide the exonuclease, SSAP, and SSB plant expression cassettes. The donor DNA template polynucleotide comprising the replacement DNA will comprise homology arms to the target DNA adjacent to the insertion site in the target genomic DNA.

In general, a donor DNA template polynucleotide including a template encoding a nucleotide change over a region of less than about 50 nucleotides is conveniently provided in the form of single-stranded DNA; larger donor templates (e.g., more than 100 nucleotides) are often conveniently provided as double-stranded DNAs. Thus in some embodiments, the donor polynucleotide is about 25 nucleotides, 50 nucleotides, 60 nucleotides, 70 nucleotides 80 nucleotides, 90 nucleotides, 100 nucleotides, 200 nucleotides, 300 nucleotides, 400 nucleotides, 500 nucleotides, 600 nucleotides, 700 nucleotides, 800 nucleotides, 900 nucleotides, 1000 nucleotides, 1200 nucleotides, 1500 nucleotides, 1800 nucleotides, 2000 nucleotides, 2500 nucleotides, 3000 nucleotides, 5000 nucleotides, 10,000 nucleotides, or more (such as about 25-200 nucleotides, 50-300 nucleotides, 100-500 nucleotides, 200-800 nucleotides, 700-2000 nucleotides, 1000-2500 nucleotides, 2000-5000 nucleotides, 4000-8000 nucleotides, or 6000-10,000 nucleotides).

Various treatments can be used for delivery (e.g., introduction) of the gene editing molecules to a plant cell.

In certain embodiments, one or more transfection-mediated methods is employed to deliver the gene editing molecules or other molecules (e.g., comprising a polynucleotide, polypeptide or combination thereof) into a eukaryotic or plant cell, e.g., through barriers such as a cell wall, a plasma membrane, a nuclear envelope, and/or other lipid bilayer. In certain embodiments, a polynucleotide-, polypeptide-, or RNP (ribonucleoprotein)-containing composition comprising the molecules are delivered directly, for example by direct contact of the composition with a plant cell. Aforementioned compositions can be provided in the form of a liquid, a solution, a suspension, an emulsion, a reverse emulsion, a colloid, a dispersion, a gel, liposomes, micelles, an injectable material, an aerosol, a solid, a powder, a particulate, a nanoparticle, or a combination thereof can be applied directly to a plant, plant part, plant cell, or plant explant (e.g., through abrasion or puncture or otherwise disruption of the cell wall or cell membrane, by spraying or dipping or soaking or otherwise directly contacting, by microinjection). For example, a plant cell or plant protoplast is soaked in a liquid genome editing molecule-containing composition. In certain embodiments, the composition is delivered using negative or positive pressure, for example, using vacuum infiltration or application of hydrodynamic or fluid pressure. In certain embodiments, the composition is introduced into a plant cell or plant protoplast, e.g., by microinjection or by disruption or deformation of the cell wall or cell membrane, for example by physical treatments such as by application of negative or positive pressure, shear forces, or treatment with a chemical or physical delivery agent such as surfactants, liposomes, or nanoparticles; see, e.g., delivery of materials to cells employing microfluidic flow through a cell-deforming constriction as described in US Published Patent Application 2014/0287509, incorporated by reference in its entirety herein. Other techniques useful for delivering the composition to a eukaryotic cell, plant cell or plant protoplast include: ultrasound or sonication; vibration, friction, shear stress, vortexing, cavitation; centrifugation or application of mechanical force; mechanical cell wall or cell membrane deformation or breakage; enzymatic cell wall or cell membrane breakage or permeabilization; abrasion or mechanical scarification (e.g., abrasion with carborundum or other particulate abrasive or scarification with a file or sandpaper) or chemical scarification (e.g., treatment with an acid or caustic agent); and electroporation.

In certain embodiments, the gene editing molecules are provided by bacterially-mediated (e.g., Agrobacterium sp., Rhizobium sp., Sinorhizobium sp., Mesorhizobium sp., Bradyrhizobium sp., Azobacter sp., Phyllobacterium sp.) transfection of the plant cell or plant protoplast with a polynucleotide encoding the genome editing molecules (e.g., RNA dependent DNA endonuclease, RNA dependent DNA binding protein, RNA dependent nickase, and/or guide RNA); see, e.g., Broothaerts et al. (2005) Nature, 433:629-633). Any of these techniques or a combination thereof are alternatively employed on the plant explant, plant part or tissue or intact plant (or seed) from which a plant cell is optionally subsequently obtained or isolated; in certain embodiments, the composition for bacterially-mediated transformation is delivered in a separate step after the plant cell has been isolated.

In certain embodiments of the methods, systems, and kits provided herein, gene editing molecules comprising the first gRNA directed to the first genomic site are introduced by biolistics, transfection, and/or electroporation and the polynucleotide encoding one or more gene editing molecules comprising the second gRNA directed to the second genomic site is introduced by bacterially-mediated transformation. In certain embodiments of the methods, systems, and kits provided herein, gene editing molecules comprising the first gRNA directed to the first genomic site and the polynucleotide encoding one or more gene editing molecules comprising the second gRNA directed to the second genomic site are introduced by biolistics, transfection, and/or electroporation.

The polynucleotides encoding one or more gene editing molecules used in the methods, systems, and kits provided herein can include a nucleotide sequence encoding a selectable marker which can be used to select a plant cell expressing the gene editing molecules. Examples of selectable markers include, but are not limited to, DNA segments that comprise restriction enzyme sites; DNA segments that encode products which provide resistance against otherwise toxic compounds including antibiotics, such as, spectinomycin, ampicillin, kanamycin, tetracycline, neomycin phosphotransferase II (NEO) and hygromycin phosphotransferase (HPT)); DNA segments that encode products which are otherwise lacking in the recipient cell (e.g., tRNA genes, auxotrophic markers); DNA segments that encode products which can be readily identified (e.g., phenotypic markers such as β-galactosidase, GUS; fluorescent proteins such as green fluorescent protein (GFP), cyan (CFP), yellow (YFP), yellow-green (mNeonGreen), red (RFP; mScarlet), and cell surface proteins); the generation of new primer sites for PCR (e.g., the juxtaposition of two DNA sequence not previously juxtaposed), the inclusion of DNA sequences not acted upon or acted upon by a restriction endonuclease or other DNA modifying enzyme, chemical, etc.; and, the inclusion of a DNA sequences required for a specific modification (e.g., methylation) that allows its identification.

Additional selectable markers include genes that confer resistance to herbicidal compounds, such as glyphosate, glufosinate, bromoxynil, imidazolinones, and 2,4-dichlorophenoxyacetate (2,4-D). See for example, Yarranton, Curr Opin Biotech (1992) 3:506-1 1; Christopherson et al, Proc. Natl. Acad. Sci. USA (1992) 89:6314-8; Yao et al, Cell (1992) 71:63-72; Reznikoff, Mol Microbiol (1992) 6:2419-22; Hu et al, Cell (1987) 48:555-66; Brown et al, Cell (1987) 49:603-12; Figge et al, Cell (1988) 52:713-22; Deuschle et al, Proc. Natl. Acad. Sci. USA (1989) 86:5400-4; Fuerst et al, Proc. Natl. Acad. Sci. USA (1989) 86:2549-53; Deuschle et al, Science (1990) 248:480-3; Gossen, (1993) Ph.D. Thesis, University of Heidelberg; Reines et al, Proc. Natl. Acad. Sci. USA (1993) 90:1917-21; Labow et al, Mol Cell Biol (1990) 10:3343-56; Zambretti et al, Proc. Natl. Acad. Sci. USA (1992) 89:3952-6; Bairn et al, Proc. Natl. Acad. Sci. USA (1991) 88:5072-6; Wyborski et al, Nucleic Acids Res (1991) 19:4647-53; Hillen and Wissman, Topics Mol Struc Biol (1989) 10:143-62; Degenkolb et al, Antimicrob Agents Chemother (1991) 35:1591-5; Kleinschmidt et al, Biochemistry (1988) 27:1094-104; Bonin, (1993) Ph.D. Thesis, University of Heidelberg; Gossen et al, Proc. Natl. Acad. Sci. USA (1992) 89:5547-51; Oliva et al, Antimicrob Agents Chemother (1992) 36:913-9; Hlavka et al, Handbook of Experimental Pharmacology, (1985) Vol. 78 (Springer-Verlag, Berlin); Gill et al, Nature (1988) 334:721-4.

Various selection procedures for the cells based on the selectable marker can be used, depending on the nature of the marker gene. In certain embodiments, use is made of a selectable marker, i.e., a marker which allows a direct selection of the cells based on the expression of the marker. A selectable marker can confer positive or negative selection and is conditional or non-conditional on the presence of external substrates (Miki et al. 2004, 107(3): 193-232). Most commonly, antibiotic or herbicide resistance genes are used as a marker, whereby selection is be performed by growing the engineered plant material on media containing an inhibitory amount of the antibiotic or herbicide to which the marker gene confers resistance. Examples of such genes are genes that confer resistance to antibiotics, such as hygromycin (hpt) and kanamycin (nptII), and genes that confer resistance to herbicides, such as phosphinothricin (bar), chlorsulfuron (als), aroA, glyphosate acetyl transferase (GAT) genes, phosphinothricin acetyl transferase (PAT) genes from Streptomyces species, and ACCase inhibitor-encoding genes. Detoxifying genes can also be used as a marker, with examples including an enzyme encoding a phosphinothricin acetyltransferase and hydroxyphenylpyruyate dioxygenase (HPPD) inhibitors.

Transformed plants and plant cells may also be identified by screening for the activities of a visible marker, typically an enzyme capable of processing a colored substrate (e.g., the β-glucuronidase, luciferase, B or CI genes).

In certain embodiment, a heterogeneous population of plant cells having a target gene edit or genome edit (or seedlings or plants grown or regenerated therefrom) is exposed to conditions permitting expression of the phenotype of interest; e.g., selection for herbicide resistance can include exposing the population of plant cells having a target gene edit or genome edit (or seedlings or plants grown or regenerated therefrom) to an amount of herbicide or other substance that inhibits growth or is toxic, allowing identification and selection of those resistant plant cells (or seedlings or plants) that survive treatment.

In some embodiments, one or more polynucleotides or vectors driving expression of one or more genome editing molecules are introduced into a plant cell. In certain embodiments, a polynucleotide vector comprises a regulatory element such as a promoter operably linked to one or more polynucleotides encoding genome editing molecules. In such embodiments, expression of these polynucleotides can be controlled by selection of the appropriate promoter, particularly promoters functional in a eukaryotic cell (e.g., plant cell); useful promoters include constitutive, conditional, inducible, and temporally or spatially specific promoters (e.g., a tissue specific promoter, a developmentally regulated promoter, or a cell cycle regulated promoter). Developmentally regulated promoters that can be used in plant cells include Phospholipid Transfer Protein (PLTP), fructose-1,6-bisphosphatase protein, NAD (P)-binding Rossmann-Fold protein, adipocyte plasma membrane-associated protein-like protein, Rieske [2Fe-2S] iron-sulfur domain protein, chlororespiratory reduction 6 protein, D-glycerate 3-kinase, chloroplastic-like protein, chlorophyll a-b binding protein 7, chloroplastic-like protein, ultraviolet-B-repressible protein, Soul heme-binding family protein, Photosystem I reaction center subunit psi-N protein, and short-chain dehydrogenase/reductase protein that are disclosed in US Patent Application Publication No. 20170121722, which is incorporated herein by reference in its entirety and specifically with respect to such disclosure. In certain embodiments, the promoter is operably linked to nucleotide sequences encoding multiple guide RNAs, wherein the sequences encoding guide RNAs are separated by a cleavage site such as a nucleotide sequence encoding a microRNA recognition/cleavage site or a self-cleaving ribozyme (see, e.g., Ferré-D′Amaré and Scott (2014) Cold Spring Harbor Perspectives Biol., 2: a003574). In certain embodiments, the promoter is an RNA polymerase III promoter operably linked to a nucleotide sequence encoding one or more guide RNAs. In certain embodiments, the RNA polymerase III promoter is a plant U6 spliceosomal RNA promoter, which can be native to the genome of the plant cell or from a different species, e.g., a U6 promoter from maize, tomato, or soybean such as those disclosed U.S. Patent Application Publication 2017/0166912, or a homologue thereof, in an example, such a promoter is operably linked to DNA sequence encoding a first RNA molecule including a Cas12a gRNA followed by an operably linked and suitable 3′ element such as a U6 poly-T terminator. In another embodiment, the RNA polymerase III promoter is a plant U3, 7SL (signal recognition particle RNA), U2, or U5 promoter, or chimerics thereof, e.g., as described in U.S. Patent Application Publication 20170166912. In certain embodiments, the promoter operably linked to one or more polynucleotides is a constitutive promoter that drives gene expression in eukaryotic cells (e.g., plant cells). In certain embodiments, the promoter drives gene expression in the nucleus or in an organelle such as a chloroplast or mitochondrion. Examples of constitutive promoters for use in plants include a CaMV 35S promoter as disclosed in U.S. Pat. Nos. 5,858,742 and 5,322,938, a rice actin promoter as disclosed in U.S. Pat. No. 5,641,876, a maize chloroplast aldolase promoter as disclosed in U.S. Pat. No. 7,151,204, and the nopaline synthase (NOS) and octopine synthase (OCS) promoters from Agrobacterium tumefaciens. In certain embodiments, the promoter operably linked to one or more polynucleotides encoding elements of a genome-editing system is a promoter from figwort mosaic virus (FMV), a RUBISCO promoter, or a pyruvate phosphate dikinase (PPDK) promoter, which is active in photosynthetic tissues. Other contemplated promoters include cell-specific or tissue-specific or developmentally regulated promoters, for example, a promoter that limits the expression of the nucleic acid targeting system to germline or reproductive cells (e.g., promoters of genes encoding DNA ligases, recombinases, replicases, or other genes specifically expressed in germline or reproductive cells). In certain embodiments, the genome alteration is limited only to those cells from which DNA is inherited in subsequent generations, which is advantageous where it is desirable that expression of the genome-editing system be limited in order to avoid genotoxicity or other unwanted effects. All of the patent publications referenced in this paragraph are incorporated herein by reference in their entirety.

Expression vectors or polynucleotides provided herein may contain a DNA segment near the 3′ end of an expression cassette that acts as a signal to terminate transcription and directs polyadenylation of the resultant mRNA and may also support promoter activity. Such a 3′ element is commonly referred to as a “3′-untranslated region” or “3′-UTR” or a “polyadenylation signal.” In some cases, plant gene-based 3′ elements (or terminators) consist of both the 3′-UTR and downstream non-transcribed sequence (Nuccio et al., 2015). Useful 3′ elements include: Agrobacterium tumefaciens nos 3′, tml 3′, tmr 3′, tms 3′, ocs 3′, and tr7 3′ elements disclosed in U.S. Pat. No. 6,090,627, incorporated herein by reference, and 3′ elements from plant genes such as the heat shock protein 17, ubiquitin, and fructose-1,6-biphosphatase genes from wheat (Triticum aestivum), and the glutelin, lactate dehydrogenase, and beta-tubulin genes from rice (Oryza sativa), disclosed in US Patent Application Publication 2002/0192813 A1. All of the patent publications referenced in this paragraph are incorporated herein by reference in their entireties.

Target plants suitable for use in the methods provided herein include plants and plant cells of any species of interest, including dicots and monocots. Plants of interest include row crop plants, fruit-producing plants and trees, vegetables, trees, and ornamental plants including ornamental flowers, shrubs, trees, groundcovers, and turf grasses. Examples of commercially important cultivated crops, trees, and plants include: alfalfa (Medicago sativa), almonds (Prunus dulcis), apples (Malus x domestica), apricots (Prunus armeniaca, P. brigantine, P. mandshurica, P. mume, P. sibirica), asparagus (Asparagus officinalis), bananas (Musa spp.), barley (Hordeum vulgare), beans (Phaseolus spp.), blueberries and cranberries (Vaccinium spp.), cacao (Theobroma cacao), canola and rapeseed or oilseed rape, (Brassica napus), carnation (Dianthus caryophyllus), carrots (Daucus carota sativus), cassava (Manihot esculentum), cherry (Prunus avium), chickpea (Cider arietinum), chicory (Cichorium intybus), chili peppers and other capsicum peppers (Capsicum annuum, C. frutescens, C. chinense, C. pubescens, C. baccatum), chrysanthemums (Chrysanthemum spp.), coconut (Cocos nucifera), coffee (Coffea spp. including Coffea arabica and Coffea canephora), cotton (Gossypium hirsutum L.), cowpea (Vigna unguiculata), cucumber (Cucumis sativus), currants and gooseberries (Ribes spp.), eggplant or aubergine (Solanum melongena), eucalyptus (Eucalyptus spp.), flax (Linum usitatissumum L.), geraniums (Pelargonium spp.), grapefruit (Citrus x paradisi), grapes (Vitus spp.) including wine grapes (Vitus vinifera), guava (Psidium guajava), hops (Humulus lupulus), hemp and cannabis (Cannabis sativa and Cannabis spp.), irises (Iris spp.), lemon (Citrus limon), lettuce (Lactuca sativa), limes (Citrus spp.), maize (Zea mays L.), mango (Mangifera indica), mangosteen (Garcinia mangostana), melon (Cucumis melo), millets (Setaria spp, Echinochloa spp, Eleusine spp, Panicum spp., Pennisetum spp.), oats (Avena sativa), oil palm (Ellis quineensis), olive (Olea europaea), onion (Allium cepa), orange (Citrus sinensis), papaya (Carica papaya), peaches and nectarines (Prunus persica), pear (Pyrus spp.), pea (Pisa sativum), peanut (Arachis hypogaea), peonies (Paeonia spp.), petunias (Petunia spp.), pineapple (Ananas comosus), plantains (Musa spp.), plum (Prunus domestica), poinsettia (Euphorbia pulcherrima), Polish canola (Brassica rapa), poplar (Populus spp.), potato (Solanum tuberosum), pumpkin (Cucurbita pepo), rice (Oryza sativa L.), roses (Rosa spp.), rubber (Hevea brasiliensis), rye (Secale cereale), safflower (Carthamus tinctorius L), sesame seed (Sesame indium), sorghum (Sorghum bicolor), soybean (Glycine max L.), squash (Cucurbita pepo), strawberries (Fragaria spp., Fragaria x ananassa), sugar beet (Beta vulgaris), sugarcanes (Saccharum spp.), sunflower (Helianthus annus), sweet potato (Ipomoea batatas), tangerine (Citrus tangerina), tea (Camellia sinensis), tobacco (Nicotiana tabacum L.), tomato (Lycopersicon esculentum), tulips (Tulipa spp.), turnip (Brassica rapa rapa), walnuts (Juglans spp. L.), watermelon (Citrulus lanatus), wheat (Tritium aestivum), and yams (Discorea spp.). In certain embodiments, the plant cell is a maize plant cell, a wheat plant cell, or a soybean plant cell

### EMBODIMENTS

The following numbered embodiments also form part of the present disclosure:

1. A method of producing a genome edited plant cell comprising:
(a) introducing into a plant cell a first guide RNA (gRNA) directed to a first genomic DNA site, a donor DNA template polynucleotide, and a polynucleotide comprising a DNA molecule encoding a second gRNA directed to a second genomic DNA site; wherein the plant cell comprises at least one Cas nuclease and/or one or more polynucleotides encoding the Cas nuclease(s); and
(b) selecting for the genome edited plant cell, wherein the genome edited plant cell comprises a first insertion of the donor DNA template polynucleotide or fragment thereof in the first genomic DNA site and a DNA modification at the second genomic DNA site comprising a second insertion, a deletion, and/or a substitution of one or more nucleotides, and wherein the second insertion does not comprise the donor DNA template polynucleotide or fragment thereof.

2. The method of embodiment 1, further comprising the step of regenerating a genome edited plant callus and/or a genome edited plant from the genome edited plant cell of step (b).

3. The method of embodiment 1 or 2, wherein the plant cell comprising the Cas nuclease comprises a polynucleotide encoding the Cas nuclease.

4. The method of embodiment 1 or 3, wherein the polynucleotide encoding the Cas nuclease expresses the first Cas nuclease in the plant cell, optionally wherein the polynucleotide encoding the Cas nuclease expresses the first Cas nuclease in the plant cell before introducing the first gRNA, donor DNA template polynucleotide, and the polynucleotide of in step (a).

5. The method of embodiment 1 or 2, wherein the Cas nuclease is provided as a ribonucleoprotein (RNP) complexed with the first gRNA and as a polynucleotide comprising a DNA molecule that encodes the Cas nuclease.

6. The method of embodiment 5, wherein the polynucleotide comprising the DNA molecule encoding the second gRNA further comprises a DNA molecule encoding a Cas nuclease which recognizes the second gRNA.

7. The method of any one of embodiments 1 to 6, wherein the plant cell comprises at least two distinct Cas nucleases, optionally wherein the two distinct Cas nucleases comprise a first Cas nuclease which recognizes the first gRNA and a second Cas nuclease which recognizes the second gRNA.

8. The method of any one of embodiments 1 to 7, wherein the Cas nucleases or one or more polynucleotides encoding the Cas nuclease(s) are introduced before and/or with the first gRNA, donor DNA template polynucleotide, and the polynucleotide comprising a DNA molecule.

9. The method of any one of embodiments 1 to 7, wherein at least two distinct Cas nucleases and/or one or more polynucleotides encoding at least one of the Cas nucleases are introduced into the plant cell in step (a), optionally wherein a first Cas nuclease which recognizes the first gRNA and a second Cas nuclease recognizes the second gRNA.

10. The method of embodiment 9, wherein the two distinct Cas nucleases comprise a first and a second Cas nuclease, wherein the first Cas nuclease recognizes the first gRNA and is introduced as an RNP and wherein a polynucleotide encoding the second gRNA and a second Cas nuclease which recognizes the second gRNA is introduced.

11. The method of any one of embodiments 1 to 10, wherein the Cas nuclease(s) comprise a Type II and/or a Type V Cas nuclease.

12. The method of any one of embodiments 1 to 11, wherein the introducing of step (a) is by bacterially-mediated transformation, optionally wherein the bacterially-mediated transformation is Agrobacterium-mediated transformation.

13. The method of any one of embodiments 1 to 11, wherein the introducing of step (a) is by biolistics, electroporation, and/or transfection.

14. The method of any one of embodiments 1 to 13, wherein the introducing of step (a) is into callus comprising the plant cell.

15. The method of any one of embodiments 1 to 13, wherein the introducing of step (a) is into an explant comprising the plant cell.

16. The method of any one of embodiments 1 to 15, wherein the donor DNA template polynucleotide lacks homology arms complementary to DNA at the first genomic site.

17. The method of embodiment 16, wherein the donor DNA template polynucleotide is integrated by non-homologous end-joining (NHEJ).

18. The method of any one of embodiments 1 to 15, wherein the donor DNA template polynucleotide comprises homology arms complementary to DNA at the first genomic site.

19. The method of embodiment 16, wherein a fragment of the is integrated at the first genomic DNA site by homology-directed repair (HDR).

20. The method of any one of embodiments 1 to 19, wherein the plant cell comprises one or more homology-dependent repair (HDR)-enhancing polypeptides, optionally wherein the HDR-enhancing polypeptides comprise a bacteriophage lambda exonuclease, a bacteriophage lambda beta single-stranded annealing protein (SSAP), and/or an E. coli single stranded binding protein (SSB).

21. The method of any one of embodiments 1 to 20, wherein the selecting comprises assaying plant cells obtained from step (a) for the first insertion of the donor DNA template polynucleotide or fragment thereof in the first genomic DNA site and/or for the DNA modification at the second genomic DNA site comprising a second insertion, a deletion, and/or a substitution of one or more nucleotides to identify a plant cell comprising the first insertion and/or the DNA modification.

22. The method of embodiment 21, wherein the assaying comprises a phenotypic assay, a nucleic acid amplification-based assay, a nucleic acid hybridization-based assay, and/or a sequencing-based assay for the insertion and/or the DNA modification.

23. The method of embodiment 21 or 22, wherein the identified plant cell is propagated or regenerated to form callus and or a plant comprising the first insertion and/or the DNA modification.

24. The method of any one of embodiments 1 to 23, wherein the plant cell is a maize plant cell, a wheat plant cell, or a soybean plant cell.

25. A plant cell genome editing system comprising a target plant cell containing a first guide RNA (gRNA) directed to a first genomic DNA site, a donor DNA template polynucleotide, a polynucleotide comprising a DNA molecule encoding a second gRNA directed to a second genomic DNA site, and at least one Cas nuclease and/or one or more polynucleotides encoding the Cas nuclease(s), wherein the system provides a genome edited plant cell comprising a first insertion of the donor DNA template polynucleotide or fragment thereof in the first genomic DNA site and a DNA modification at the second genomic DNA site comprising a second insertion, a deletion, and/or a substitution of one or more nucleotides, wherein the second insertion does not comprise the donor DNA template polynucleotide or fragment thereof.

26. The system of embodiment 25, wherein the system provides for recovery of the genome edited plant cell from the target plant cell in a single selection.

27. The system of embodiment 25 or 26, wherein the genome edited plant cell is obtained from the target plant cell without first selecting for a plant cell comprising only the insertion at the first genomic site or only the modification at the second genomic site.

28. The system of embodiment 25, 26, or 27, wherein the Cas nuclease is provided as a ribonucleoprotein (RNP) complexed with the first gRNA and as a polynucleotide comprising a DNA molecule that encodes the Cas nuclease.

29. The system of any one of embodiments 25 to 28, wherein the plant cell comprises at least two distinct Cas nucleases, optionally wherein the two distinct Cas nucleases comprise a first Cas nuclease which recognizes the first gRNA and a second Cas nuclease which recognizes the second gRNA.

30. The system of embodiment 31, wherein at least two distinct Cas nucleases and/or one or more polynucleotides encoding at least one of the Cas nucleases are introduced into the plant cell in step (a), optionally wherein a first Cas nuclease which recognizes the first gRNA and a second Cas nuclease recognizes the second gRNA.

31. The system of embodiment 29 or 30, wherein the two distinct Cas nucleases comprise a first and a second Cas nuclease, wherein the first Cas nuclease recognizes the first gRNA and is introduced as an RNP and wherein a polynucleotide encoding the second gRNA and a second Cas nuclease which recognizes the second gRNA is introduced.

32. The system of any one of embodiments 25 to 31, wherein the Cas nuclease(s) comprise a Type II and/or a Type V Cas nuclease.

33. The system of any one of embodiments 25 to 32, wherein the donor DNA template polynucleotide lacks homology arms complementary to DNA at the first genomic site.

34. The system of embodiment 33, wherein the donor DNA template polynucleotide is integrated at the first genomic DNA site by non-homologous end-joining (NHEJ).

35. The system of any one of embodiments 25 to 32, wherein the donor DNA template polynucleotide comprises homology arms complementary to DNA at the first genomic site.

36. The system of embodiment 35, wherein a fragment of the donor DNA template polynucleotide is integrated at the first genomic DNA site by homology-directed repair (HDR).

37. The system of any one of embodiments 25 to 36, wherein the target plant cell further comprises one or more homology-dependent repair (HDR)-enhancing polypeptides, optionally wherein the HDR-enhancing polypeptides comprise a bacteriophage lambda exonuclease, a bacteriophage lambda beta single-stranded annealing protein (SSAP), and/or an E. coli single stranded binding protein (SSB).

38. The system of any one of embodiments 25 to 37, further comprising an assay to identify the genome edited plant cell.

39. The system of embodiment 38, wherein the genome edited plant cell is selected from a population of the target plant cells with the assay.

40. The system of embodiment 39, wherein the assay comprises a phenotypic assay, a nucleic acid amplification-based assay, a nucleic acid hybridization-based assay, and/or a sequencing-based assay for the insertion and/or the DNA modification.

41. The system of any one of embodiments 25 to 40, wherein the system further comprises a plant tissue culture system for regenerating callus or a plant from the genome edited plant cell.

42. The system of any one of embodiments 25 to 41, wherein the plant cell is a maize plant cell, a wheat plant cell, or a soybean plant cell.

43. A kit comprising:
(a) a first guide RNA (gRNA) directed to a first genomic DNA site in a plant cell and a donor DNA template polynucleotide; and,
(b) a polynucleotide comprising a DNA molecule encoding a second gRNA directed to a second genomic DNA site in a plant cell.

44. The kit of embodiment 43, wherein the kit further comprises at least one Cas nuclease that recognizes at least one of the gRNAs.

45. The kit of embodiment 44, wherein the first gRNA is complexed with a Cas nuclease as a ribonuclear protein (RNP).

46. The kit of embodiment 43, 44, or 45, wherein the DNA molecule encodes a Cas nuclease.

47. The kit of any one of embodiments 44, 45, or 46, wherein the Cas nuclease in the RNP is distinct from the Cas nuclease encoded by the DNA molecule.

48. The kit of any one of embodiments 44 to 47, wherein the Cas nuclease(s) comprise a Type II and/or a Type V Cas nuclease.

49. The kit of any one of embodiments 43 to 48, wherein the donor DNA template polynucleotide lacks homology arms complementary to DNA at the first genomic site.

50. The kit of embodiment 49, wherein the donor DNA template polynucleotide comprises homology arms complementary to DNA at the first genomic site.

51. The kit of any one of embodiments 43 to 50, wherein the kit further comprises a plant cell containing the first and the second genomic sites, optionally wherein the plant cell is a maize plant cell, a wheat plant cell, or a soybean plant cell.

### EXAMPLES

### Example 1

Immature embryos from a maize line ubiquitously expressing a Cas nuclease in the B104 background were bombarded with gold particles coated with: (i) guide RNAs targeting the promoters of the maize ARGOS8 gene (B73: Zm00001d038075; B104: Zm00007a00003399), PIP2-5 gene (B73: Zm00001d003006; B104: Zm00007a00024679), and NF-YB16 gene (B73: Zm00001d022099; B104: Zm00007a00029813); (ii) a donor DNA template polynucleotide comprising a double-stranded 5′ phosphorylated oligonucleotide with 2 phosphorothioate bonds at both ends with a triple enhancer sequence (GTAAGCGCTTACGTAAGCGCTTACGTAAGCGCTTAC; SEQ ID NO: 1; U.S. Pat. No. 11,198,885, incorporated herein by reference in its entirety); and (iii) a plasmid encoding the phosphinothricin acetyl transferase (pat) resistance gene, a fluorescent protein, and two (2) gRNAs targeting the coding sequences of maize gene HB54 (Zm00001d005951/Zm00007a00014428) and bZIP100 (Zm00001d044940/Zm00007a00041818) driven by the TaU3 and OsU3 RNA polymerase III promoters, respectively.

Through standard tissue culture practices, with selection for pat-mediated herbicide resistance, plants were regenerated. Leaf tissue was taken from regenerated plants for gDNA extraction to check for the presence of donor DNA template polynucleotide oligo insertions in the targeted ARGOS8, PIP2-5, and NF-YB16 promoters and small indels in the “knock-out” (KO) targets (HB54 and bZIP100). 37 plants were regenerated from 12 transformation events, and 21 (57%) had an insertion in at least one promoter, and all of these had an insertion in the PIP2-5 promoter.

Of these 21 plants, two (10%) had homozygous or biallelic CRISPR/Cas-induced frameshift mutations in Zm00007a00014428 (Table 1). In 6 other plants, some level of editing (1.7-33% of reads) could be seen, suggesting that in the next generation after selfing homozygotes could be obtained (Table 1). The oligonucleotide was not inserted at the gRNA target sites of KO targets Zm00007a00014428 and Zm00007a00041818 in any of the edited plants. Table 1 (below) shows the editing frequencies (% of reads showing an indel at the gRNA target site) at the two KO targets in all plants showing insertions in at least one promoter and editing at any of the two KO targets.

TABLE 1

| Plant | HB54 | bZIP100 |
|---|---|---|
| ZM205-3A | 33.3% | 33.9% |
| ZM212-1A | 98.5% (52% insertion of T, | 13.1% |
| | 46% insertion of C) | |
| ZM212-1B | 3.3% | 1.2% |
| ZM212-1C | 4.4% | 2.8% |
| ZM212-1D | 1.7% | 0.7% |
| ZM212-7A | 99.5% (99.5% insertion of A) | 12.3% |
| ZM212-7B | 15.0% | 18.2% |
| ZM212-7C | 14.1% | 11.9% |

The breadth and scope of the present disclosure should not be limited by any of the above-described examples.

What is claimed is:

1. A method of producing a genome edited plant cell comprising:
(a) introducing into a plant cell a first guide RNA (gRNA) directed to a first genomic DNA site, a donor DNA template polynucleotide, and a polynucleotide comprising a DNA molecule encoding a second gRNA directed to a second genomic DNA site; wherein the plant cell comprises at least one Cas nuclease and/or one or more polynucleotides encoding the Cas nuclease(s); and
(b) selecting for the genome edited plant cell, wherein the genome edited plant cell comprises a first insertion of the donor DNA template polynucleotide or fragment thereof in the first genomic DNA site and a DNA modification at the second genomic DNA site comprising a second insertion, a deletion, and/or a substitution of one or more nucleotides, and wherein the second insertion does not comprise the donor DNA template polynucleotide or fragment thereof.

2. The method of claim 1, further comprising the step of regenerating a genome edited plant callus and/or a genome edited plant from the genome edited plant cell of step (b).

3. The method of claim 1, wherein the plant cell comprising the Cas nuclease comprises a polynucleotide encoding the Cas nuclease.

4. The method of claim 1, wherein the polynucleotide encoding the Cas nuclease expresses the first Cas nuclease in the plant cell, optionally wherein the polynucleotide encoding the Cas nuclease expresses the first Cas nuclease in the plant cell before introducing the first gRNA, donor DNA template polynucleotide, and the polynucleotide of in step (a).

5. The method of claim 1, wherein the Cas nuclease is provided as a ribonucleoprotein (RNP) complexed with the first gRNA and as a polynucleotide comprising a DNA molecule that encodes the Cas nuclease.

6. The method of claim 5, wherein the polynucleotide comprising the DNA molecule encoding the second gRNA further comprises a DNA molecule encoding a Cas nuclease which recognizes the second gRNA.

7. The method of claim 1, wherein the plant cell comprises at least two distinct Cas nucleases, optionally wherein the two distinct Cas nucleases comprise a first Cas nuclease which recognizes the first gRNA and a second Cas nuclease which recognizes the second gRNA.

8. The method of claim 1, wherein the Cas nucleases or one or more polynucleotides encoding the Cas nuclease(s) are introduced before and/or with the first gRNA, donor DNA template polynucleotide, and the polynucleotide comprising a DNA molecule.

9. The method of claim 1, wherein at least two distinct Cas nucleases and/or one or more polynucleotides encoding at least one of the Cas nucleases are introduced into the plant cell in step (a), optionally wherein a first Cas nuclease which recognizes the first gRNA and a second Cas nuclease recognizes the second gRNA.

10. The method of claim 9, wherein the two distinct Cas nucleases comprise a first and a second Cas nuclease, wherein the first Cas nuclease recognizes the first gRNA and is introduced as an RNP and wherein a polynucleotide encoding the second gRNA and a second Cas nuclease which recognizes the second gRNA is introduced.

11. The method of claim 1, wherein the Cas nuclease(s) comprise a Type II and/or a Type V Cas nuclease.

12. The method of claim 1, wherein the introducing of step (a) is by bacterially-mediated transformation, optionally wherein the bacterially-mediated transformation is Agrobacterium-mediated transformation.

13. The method of claim 1, wherein the introducing of step (a) is by biolistics, electroporation, and/or transfection.

14. The method of claim 1, wherein the introducing of step (a) is into callus comprising the plant cell.

15. The method of claim 1, wherein the introducing of step (a) is into an explant comprising the plant cell.

16. The method of claim 1, wherein the donor DNA template polynucleotide lacks homology arms complementary to DNA at the first genomic site.

17. The method of claim 16, wherein the donor DNA template polynucleotide is integrated by non-homologous end-joining (NHEJ).

18. The method of claim 1, wherein the donor DNA template polynucleotide comprises homology arms complementary to DNA at the first genomic site.

19. The method of claim 18, wherein a fragment of the donor DNA template polynucleotide is integrated at the first genomic DNA site by homology-directed repair (HDR).

20. The method of any one of claims 1 to 19, wherein the plant cell comprises one or more homology-dependent repair (HDR)-enhancing polypeptides, optionally wherein the HDR-enhancing polypeptides comprise a bacteriophage lambda exonuclease, a bacteriophage lambda beta single-stranded annealing protein (SSAP), and/or an E. coli single stranded binding protein (SSB).

21. The method of any one of claims 1 to 19, wherein the selecting comprises assaying plant cells obtained from step (a) for the first insertion of the donor DNA template polynucleotide or fragment thereof in the first genomic DNA site and/or for the DNA modification at the second genomic DNA site comprising a second insertion, a deletion, and/or a substitution of one or more nucleotides to identify a plant cell comprising the first insertion and/or the DNA modification.

22. The method of claim 21, wherein the assaying comprises a phenotypic assay, a nucleic acid amplification-based assay, a nucleic acid hybridization-based assay, and/or a sequencing-based assay for the insertion and/or the DNA modification.

23. The method of claim 21, wherein the identified plant cell is propagated or regenerated to form callus and or a plant comprising the first insertion and/or the DNA modification.

24. The method of any one of claims 1 to 19, wherein the plant cell is a maize plant cell, a wheat plant cell, or a soybean plant cell.

25. A plant cell genome editing system comprising a target plant cell containing a first guide RNA (gRNA) directed to a first genomic DNA site, a donor DNA template polynucleotide, a polynucleotide comprising a DNA molecule encoding a second gRNA directed to a second genomic DNA site, and at least one Cas nuclease and/or one or more polynucleotides encoding the Cas nuclease(s), wherein the system provides a genome edited plant cell comprising a first insertion of the donor DNA template polynucleotide or fragment thereof in the first genomic DNA site and a DNA modification at the second genomic DNA site comprising a second insertion, a deletion, and/or a substitution of one or more nucleotides, wherein the second insertion does not comprise the donor DNA template polynucleotide or fragment thereof.

26. The system of claim 25, wherein the system provides for recovery of the genome edited plant cell from the target plant cell in a single selection.

27. The system of claim 25, wherein the genome edited plant cell is obtained from the target plant cell without first selecting for a plant cell comprising only the insertion at the first genomic site or only the modification at the second genomic site.

28. The system of claim 25, wherein the Cas nuclease is provided as a ribonucleoprotein (RNP) complexed with the first gRNA and as a polynucleotide comprising a DNA molecule that encodes the Cas nuclease.

29. The system of claim 25, wherein the plant cell comprises at least two distinct Cas nucleases, optionally wherein the two distinct Cas nucleases comprise a first Cas nuclease which recognizes the first gRNA and a second Cas nuclease which recognizes the second gRNA.

30. The system of claim 29, wherein at least two distinct Cas nucleases and/or one or more polynucleotides encoding at least one of the Cas nucleases are introduced into the plant cell in step (a), optionally wherein a first Cas nuclease which recognizes the first gRNA and a second Cas nuclease recognizes the second gRNA.

31. The system of claim 29, wherein the two distinct Cas nucleases comprise a first and a second Cas nuclease, wherein the first Cas nuclease recognizes the first gRNA and is introduced as an RNP and wherein a polynucleotide encoding the second gRNA and a second Cas nuclease which recognizes the second gRNA is introduced.

32. The system of claim 25, wherein the Cas nuclease(s) comprise a Type II and/or a Type V Cas nuclease.

33. The system of claim 25, wherein the donor DNA template polynucleotide lacks homology arms complementary to DNA at the first genomic site.

34. The system of claim 33, wherein the donor DNA template polynucleotide is integrated at the first genomic DNA site by non-homologous end-joining (NHEJ).

35. The system of claim 25, wherein the donor DNA template polynucleotide comprises homology arms complementary to DNA at the first genomic site.

36. The system of claim 35, wherein a fragment of the donor DNA template polynucleotide is integrated at the first genomic DNA site by homology-directed repair (HDR).

37. The system of claim 25, wherein the target plant cell further comprises one or more homology-dependent repair (HDR)-enhancing polypeptides, optionally wherein the HDR-enhancing polypeptides comprise a bacteriophage lambda exonuclease, a bacteriophage lambda beta single-stranded annealing protein (SSAP), and/or an E. coli single stranded binding protein (SSB).

38. The system of claim 25, further comprising an assay to identify the genome edited plant cell.

39. The system of claim 25, wherein the genome edited plant cell is selected from a population of the target plant cells with the assay.

40. The system of claim 39, wherein the assay comprises a phenotypic assay, a nucleic acid amplification-based assay, a nucleic acid hybridization-based assay, and/or a sequencing-based assay for the insertion and/or the DNA modification.

41. The system of claim 39, wherein the system further comprises a plant tissue culture system for regenerating callus or a plant from the genome edited plant cell.

42. The system of claim 25, wherein the plant cell is a maize plant cell, a wheat plant cell, or a soybean plant cell.

43. A kit comprising:
(a) a first guide RNA (gRNA) directed to a first genomic DNA site in a plant cell and a donor DNA template polynucleotide; and,
(b) a polynucleotide comprising a DNA molecule encoding a second gRNA directed to a second genomic DNA site in a plant cell.

44. The kit of claim 43, wherein the kit further comprises at least one Cas nuclease that recognizes at least one of the gRNAs.

45. The kit of claim 44, wherein the first gRNA is complexed with a Cas nuclease as a ribonuclear protein (RNP).

46. The kit of claim 43 or 44, wherein the DNA molecule encodes a Cas nuclease.

47. The kit of claim 46, wherein the Cas nuclease in the RNP is distinct from the Cas nuclease encoded by the DNA molecule.

48. The kit of claim 44, wherein the Cas nuclease(s) comprise a Type II and/or a Type V Cas nuclease.

49. The kit of claim 43, wherein the donor DNA template polynucleotide lacks homology arms complementary to DNA at the first genomic site.

50. The kit of claim 43, wherein the donor DNA template polynucleotide comprises homology arms complementary to DNA at the first genomic site.

51. The kit of claim 43, wherein the kit further comprises a plant cell containing the first and the second genomic sites, optionally wherein the plant cell is a maize plant cell, a wheat plant cell, or a soybean plant cell.
